(19) United States
(12) Patent Application Publication
(10) Pub. No.: US 2021/0290568 A1
(43) Pub. Date: Sep. 23, 2021

(54) INHALABLE FORMULATION OF A SOLUTION CONTAINING LEVALBUTEROL TARTRATE

(71) Applicants: Cai Gu Huang, Shrewsbury, MA (US); Hua Huang, Shanghai (CN)

(72) Inventors: Cai Gu Huang, Shrewsbury, MA (US); Hua Huang, Shanghai (CN)

(73) Assignee: Cai Gu Huang, Shrewsbury, MA (US)

(21) Appl. No.: 17/205,660

(22) Filed: Mar. 18, 2021

Related U.S. Application Data
(60) Provisional application No. 62/991,601, filed on Mar. 19, 2020.

(51) Int. Cl. A61K 31/137; A61K 9/00; A61K 47/18; A61M 15/00; A61M 11/02

(52) U.S. Cl. CPC A61K 31/137; A61K 9/0078; A61K 47/186; A61M 15/0021; A61M 15/0073; A61M 11/02; A61K 47/183

(57) ABSTRACT

The present invention discloses a liquid pharmaceutical preparation and a method for administering a pharmaceutical preparation by nebulizing the pharmaceutical preparation in an inhaler. The propellant-free pharmaceutical preparation comprises: (a) active levalbuterol or a salt thereof, such as levalbuterol tartrate; (b) a pharmacologically acceptable preservative; (c) a pharmacologically acceptable stabilizer, (d) a solvent, and optionally (e) other pharmacologically acceptable additives.

### PRIORITY STATEMENT

This application claims the benefit of the filing date of U.S. Provisional Patent Application No. 62/991,601, filed on Mar. 19, 2020, the contents of which are incorporated herein by reference in its entirety.

### FIELD OF THE INVENTION

Levalbuterol tartrate (R-AS), chemically 4-[(1R)-2-(tert-butylamino)-1-hydroxyethyl]-2-(hydroxymethyl)phenol; (2R,3R)-2,3-dihydroxybutanedioic acid, has the following chemical structure:

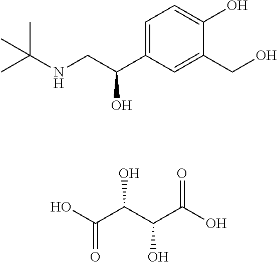 [SMILES: CC(C)(C)NC[C@H](O)c1ccc(O)c(CO)c1 ; O=C(O)[C@H](O)[C@@H](O)C(=O)O]

Levalbuterol tartrate is the tartrate salt form of levalbuterol, the R-enantiomer of the short-acting beta-2 adrenergic receptor agonist albuterol, with bronchodilator activity. Levalbuterol selectively binds to beta-2 adrenergic receptors in bronchial smooth muscle, thereby activating intracellular adenyl cyclase, an enzyme that catalyzes the conversion of adenosine triphosphate (ATP) to cyclic-3′,5′-adenosine monophosphate (cAMP). Increased cAMP levels cause relaxation of bronchial smooth muscle, relieve bronchospasms, improve mucociliary clearance, and inhibit the release of mediators of immediate hypersensitivity from cells, such as mast cells.

Levalbuterol and pharmaceutically acceptable salts thereof, such as levalbuterol tartrate, can provide therapeutic benefits for the treatment of asthma and chronic obstructive pulmonary disease, including chronic bronchitis and emphysema.

The present invention relates to a propellant-free inhalable formulation of levalbuterol or pharmaceutically acceptable salts thereof, such as levalbuterol tartrate, dissolved in water, which can be administered by a soft mist inhaler, and propellant-free inhalable aerosols resulting therefrom.

The pharmaceutical formulations disclosed in the current invention are particularly suitable for soft mist inhalation, which have good lung deposition (typically up to 55-60%), compared to dry powder inhalation methods. Furthermore, the liquid inhalation formulations are advantageous compared to dry powder inhalation. In particular, administration by dry powder inhalation is more difficult, particularly for children and elderly patients.

The present invention relates to a novel approach to more effectively and selectively deliver levalbuterol or pharmaceutically acceptable salts thereof, such as levalbuterol tartrate, to the lungs by soft mist inhalation as compared to dry powder inhalation.

### SUMMARY OF THE INVENTION

A novel, surprising approach to a more effective and selective method of delivering levalbuterol or pharmaceutically acceptable salts thereof, such as levalbuterol tartrate, to the lungs has been found, which more effectively deposits the active ingredient in the lungs. The novel method of the current invention presents clear and significant clinical benefits, including higher efficacy and fewer adverse effects.

The present invention relates to pharmaceutical formulations of levalbuterol or pharmaceutically acceptable salts or solvates thereof, such as levalbuterol tartrate, which can be administered by soft mist inhalation. The pharmaceutical formulations according to the current invention meet high quality standards.

One aspect of the present invention is to provide an aqueous pharmaceutical formulation containing levalbuterol or pharmaceutically acceptable salts thereof, such as levalbuterol tartrate, which meets high standards and is able to achieve an optimum atomization effect with administration using a soft mist inhaler. It is desirable that the active ingredient in the formulation be pharmaceutically stable for a storage period of a few months or years, such as about 1-6 months, about one year, or about three years.

Another aspect of the present invention is providing propellant-free formulations of solutions containing levalbuterol or pharmaceutically acceptable salts thereof, such as levalbuterol tartrate, which are nebulized under pressure using an inhaler, such as a soft mist inhaler device. In one embodiment, the formulation delivered by the inhaler is an aerosol having particle sizes that reproducibly fall within a specified and desirable range.

Another aspect of the present invention is providing stable pharmaceutical formulations of aqueous solutions containing levalbuterol or pharmaceutically acceptable salts thereof, such as levalbuterol tartrate, and excipients which can be administered by soft mist inhalation devices.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 shows a longitudinal section through the atomizer in the stressed state.

FIG. 2 shows a counter element of the atomizer.

FIG. 3 shows an HPLC trace demonstrating the relative retention times of impurities measured in stability experiments at 0 days and 1 month duration in Example 7.

FIG. 4 shows an HPLC trace demonstrating the relative retention times of impurities measured in stability experiments at 3 months duration in Example 7.

The use of identical or similar reference numerals in different figures denotes identical or similar features.

### DETAILED DESCRIPTION OF THE INVENTION

For purposes of describing the invention, reference now will be made in detail to embodiments and/or methods of the invention, one or more examples of which are illustrated in or with the drawings. Each example is provided by way of explanation of the invention, not limitation of the invention. In fact, it will be apparent to those skilled in the art that various modifications and variations can be made in the present invention without departing from the scope or spirit of the invention. For instance, features or steps illustrated or described as part of one embodiment, can be used with another embodiment or steps to yield a still further embodiments or methods. Thus, it is intended that the present invention covers such modifications and variations as come within the scope of the appended claims and their equivalents.

It is desirable to achieve better delivery and distribution of active substances in the lungs for the treatment of respiratory diseases. Furthermore, it is desirable to maximize deposition of the drug in the lungs when the drug is delivered by inhalation.

A device for the propellant-free administration of a metered amount of a liquid pharmaceutical composition for inhalation is described in detail, for example, in US20190030268, the disclosure of which is incorporated herein by reference. Such devices can significantly increase the lung deposition of inhalable drugs.

In an embodiment, the aforementioned inhalers nebulize a small amount of the pharmaceutical liquid formulation within a few seconds into an aerosol that is suitable for therapeutic inhalation.

Soft mist inhaler devices for use with the aqueous pharmaceutical formulation of the present invention are those in which an amount of less than about 70 microliters of pharmaceutical solution can be nebulized in one puff, such as less than about 30 microliters, or such as less than about 15 microliters, so that the inhalable part of aerosol corresponds to a therapeutically effective quantity. In an embodiment, the average particle size of the aerosol formed from one puff is less than about 15 microns, such as less than about 10 microns.

In soft mist inhalers that can be used with the invention, the pharmaceutical solution is stored in a reservoir. In an embodiment, the pharmaceutical formulations of the invention do not contain any ingredients which might interact with the inhaler and affect the pharmaceutical quality of the formulation or of the aerosol produced. In an embodiment, the pharmaceutical formulations of the invention are stable when stored and are capable of being administered directly.

In an embodiment, the pharmaceutical formulations of the current invention contain additives, such as the disodium salt of edetic acid (sodium edetate), to reduce the incidence of spray anomalies and to stabilize the pharmaceutical formulations. In an embodiment, the pharmaceutical formulations of the current invention have a minimum concentration of sodium edetate.

Therefore, one aspect of the present invention is to provide an aqueous pharmaceutical formulation containing levalbuterol or pharmaceutically acceptable salts thereof, such as levalbuterol tartrate, which meets high standards to achieve an optimum atomization effect with administration using the inhalers mentioned hereinbefore. In one embodiment, the active substance in the pharmaceutical formulation is stable, and the pharmaceutical formulation has a storage time of some years, for example about one year, or about three years.

Another aspect of the current invention is to provide propellant-free formulations containing levalbuterol or pharmaceutically acceptable salts thereof, such as levalbuterol tartrate, which may be present in a solution. In an embodiment, the formulations are nebulized under pressure using an inhaler, such as a soft mist inhaler, wherein the resulting aerosol produced by the inhaler falls reproducibly within a specified range of particle size.

Another aspect of the invention is to provide an aqueous pharmaceutical formulation as a solution containing levalbuterol or pharmaceutically acceptable salts thereof, such as levalbuterol tartrate, and inactive excipients which can be administered by inhalation.

According to the invention, any pharmaceutically acceptable salts or solvates of levalbuterol may be used for the formulations. In one aspect of the invention, the pharmaceutically acceptable salt or solvate of levalbuterol is levalbuterol tartrate.

In an embodiment, the levalbuterol tartrate is dissolved in a solvent. In one embodiment, the solvent is water.

The concentration of levalbuterol tartrate or its pharmaceutically acceptable salt in the finished pharmaceutical preparation depends on the therapeutic effects and can be determined by a person of ordinary skill in the art. In an embodiment, the concentration of levalbuterol tartrate in the formulation is between about 20 mg/100 g and about 10 g/100 g, more specifically between about 200 mg/100 g and about 500 mg/100 g.

In one aspect of the invention, the pharmaceutical formulation includes a stabilizer or complexing agent. In one embodiment, the formulation includes edetic acid (EDTA) or a salt thereof, such as edetate disodium, edetate disodium dihydrate, or citric acid, as a stabilizer or complexing agent. In an embodiment, the formulation contains edetic acid and/or a salt thereof.

A complexing agent is a molecule which is capable of entering into complex bonds. In an embodiment, complexing agents have the effect of complexing cations. The concentration of the stabilizers or complexing agents is about 5 mg/100 g to about 100 mg/100 g. In another embodiment, the concentration of the stabilizers or complexing agents is about 5 mg/100 g to about 25 mg/100 g.

In one embodiment, the levalbuterol tartrate is present in solution.

In another embodiment, all the ingredients of the formulation are present in solution.

The formulations may further include additives. The term “additives,” as used herein, means any pharmacologically acceptable and/or therapeutically useful substance that is not an active substance, but that can be formulated together with the active substance to improve the qualities of the formulation. In an embodiment, additives have no appreciable pharmacological effect in the context of the desired therapy.

The additives include, but are not limited to, for example, other stabilizers, complexing agents, antioxidants, surfactants, preservatives which prolong the shelf life of the finished pharmaceutical formulation, vitamins and/or other additives known in the art.

In one aspect of the invention, the formulations includes an acid or a base as a pH adjusting agent. In an embodiment, the pH adjusting agent is an acid, such as citric acid and/or a salt thereof. In another embodiment, the pH adjusting agent is a base, such as sodium hydroxide.

Other comparable pH adjusting agents can be used in the present invention. Other pH adjusting agents include, but are not limited to, hydrochloric acid, sodium citrate, and sodium hydroxide.

Adjusting the pH may provide better stability of active substances. In an embodiment, the pH ranges from about 3.0 to about 6.0. In another embodiment, the pH ranges from about 3.0 to about 5.0.

In one aspect of the invention, the formulation further comprises a suitable preservative to protect the formulation from contamination with pathogenic bacteria. In one embodiment, the preservative comprises benzalkonium chloride, benzoic acid, or sodium benzoate. In an embodiment, the pharmaceutical formulations contain only benzalkonium chloride as a preservative. In one embodiment, the amount of preservative ranges from about 10 mg/100 g to about 100 mg/100 g.

To produce the propellant-free aerosols according to the invention, the pharmaceutical formulations containing levalbuterol or its pharmaceutically acceptable salts, such as levalbuterol tartrate, may be used with a soft mist inhaler of the kind described herein.

The soft mist inhaler disclosed in U.S. 2019/0030268, which is incorporated by reference, is an example of an inhaler that is suitable for use with the formulations of the present invention.

The inhalation device can be carried anywhere by the patient, having a cylindrical shape and convenient size of less than about 8 cm to about 18 cm long, and about 2.5 cm to about 5 cm wide. In an embodiment, the inhalation device sprays a defined volume of the pharmaceutical formulation out through small nozzles at high pressures, so as to produce inhalable aerosols.

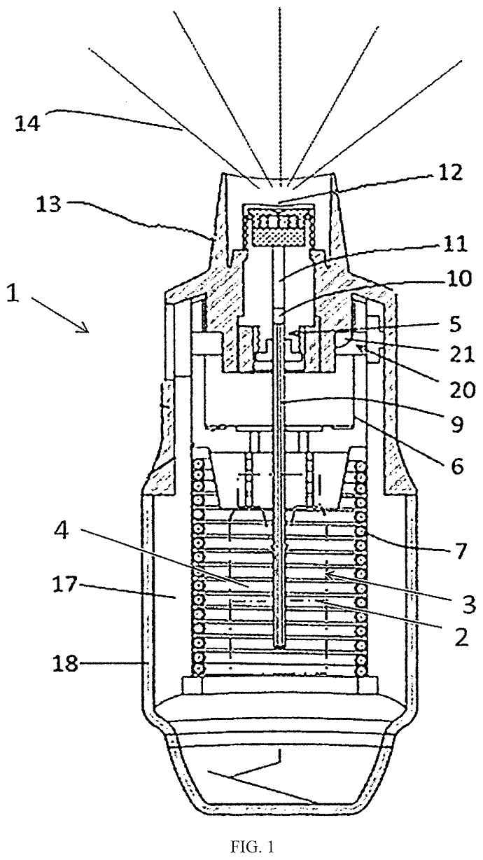

FIG. 1 shows a longitudinal section through the atomizer in the stressed state. In an embodiment, the atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, and an inside part 17.

The inhalation atomizer 1 comprising the blocking function and the counter described above for spraying a medicament fluid 2 is demonstrated in the FIG. 1 in the stressed state. The atomizer 1 described is a propellant-free portable inhaler.

For the typical atomizer 1 comprising the blocking function and the counter described above, an aerosol 14 that can be inhaled by a patient is generated through the atomization of the fluid 2. The pharmaceutical formulation is typically administered at least once a day, and in one embodiment, multiple times a day, at predetermined time intervals, according to how seriously the illness affects the patient. A person of ordinary skill in the art would be able to determine the frequency with which the pharmaceutical formulation is to be administered.

In an embodiment, the atomizer 1 described above has a substitutable and insertable vessel 3, which contains the pharmaceutical formulation. Therefore, a reservoir for holding the fluid 2 is formed in the vessel 3. Specifically, the pharmaceutical formulation is located in the fluid compartment 4 formed by a collapsible bag in the vessel 3.

In an embodiment, the amount of fluid 2 for the atomizer 1 can provide an adequate amount for a patient, such as up to 200 doses. In an embodiment, vessel 3 has a volume of about 2 to about 10 ml. A pressure generator 5 in the atomizer 1 is used to deliver and atomize the fluid 2, specifically in a predetermined dosage amount. The fluid 2 is released and sprayed in individual doses, such as from about 5 to about 30 microliters.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 could be separated from the atomizer 1 for substitution.

In an embodiment, when drive spring 7 is stressed in an axial direction, the delivering tube 9, the vessel 3, and the holder 6 will be shifted downwards. The fluid 2 will then be sucked into the pressure room 11 through the delivering tube 9 and the non-return valve 10.

In an embodiment, after releasing the holder 6, the stress is eased. During this process, the delivering tube 9 and closed non-return valve 10 are shifted back upward by releasing the drive spring 7. The fluid 2 is under pressure in the pressure room 11. The fluid 2 is pushed through the nozzle 12 and atomized into an aerosol 14 by the pressure. A patient may inhale the aerosol 14 through the mouthpiece 13, while the air is sucked into the mouthpiece 13 through air inlets 15.

In an embodiment, the atomizer described above has an upper shell 16 and an inside part 17, which may be rotated relative to the upper shell 16. A lower shell 18 is manually operable to attach onto the inside part 17. The lower shell 18 may be separated from the atomizer 1 so that the vessel 3 can be substituted and inserted.

In an embodiment, the atomizer 1 described above may have a lower shell 18, which carries the inside part 17, and may be rotatable relative to the upper shell 16. As a result of rotation and engagement between the upper unit 17 and the holder 6, through a gear 20, the holder 6 is axially moved counter to the force of the drive spring 7, and the drive spring 7 is stressed.

In an embodiment, in the stressed state, the vessel 3 is shifted downwards and reaches a final position, which is demonstrated in FIG. 1. The drive spring 7 is stressed under this final position, and the holder 6 is clasped. The vessel 3 and the delivering tube 9 are prevented from moving upwards so that the drive spring 7 is stopped from easing.

In an embodiment, in the stressed state, the atomizing process occurs after releasing the holder 6. The vessel 3, the delivering tube 9, and the holder 6 are shifted back by the drive spring 7 to the beginning position. This is referred to as major shifting. While the major shifting occurs, the non-return valve 10 is closed and the fluid 2 is under pressure in the pressure room 11 by the delivering tube 9, and then the fluid 2 is pushed out and atomized by the pressure.

In an embodiment, the atomizer 1 described above may have a clamping function. During clamping, the vessel 3 may perform a lifting shift for the withdrawal of the fluid 2 during the atomizing process. The gear 20 has sliding surfaces 21 on the upper shell 16 and/or on the holder 6, which could make holder 6 axially move when the holder 6 is rotated relative to the upper shell 16.

In an embodiment, the holder 6 is not blocked for too long and can undergo the major shifting. The fluid 2 is pushed out and atomized.

In an embodiment, when the holder 6 is in the clamping position, the sliding surfaces 21 move out of engagement. The gear 20 releases the holder 6 for the opposite axial shift.

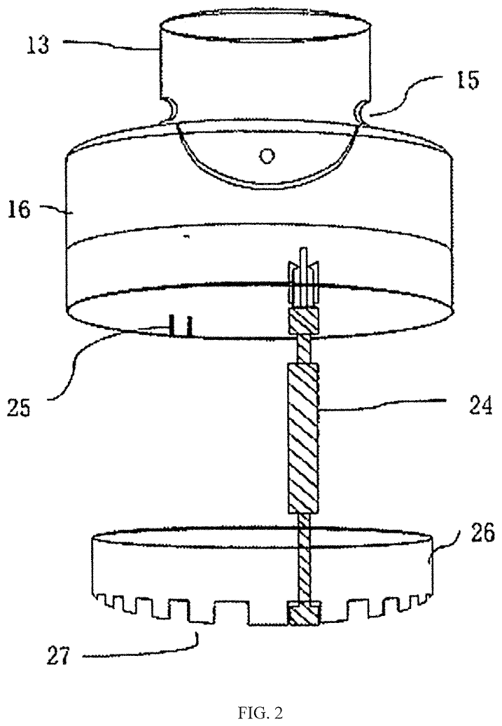

In an embodiment, the atomizer 1 includes a counter element shown in FIG. 2. The counter element has a worm 24 and a counter ring 26. In an embodiment, the counter ring 26 is circular and has dentate part at the bottom. The worm 24 has upper and lower end gears. The upper end gear contacts an upper shell 16. The upper shell 16 has an inside bulge 25. When the atomizer 1 is employed, the upper shell 16 rotates, and when the bulge 25 passes through the upper end gear of the worm 24, the worm 24 is driven to rotate. The rotation of the worm 24 drives the rotation of the counter ring 26 through the lower end gear. This results in the counting effect.

In an embodiment, the locking mechanism comprises at least two protrusions. Protrusion A is located on the outer wall of the lower unit of the inside part. Protrusion B is located on the inner wall of the counter. The lower unit of the inside part is nested in the counter. In an embodiment, the counter may rotate relative to the lower unit of the inside part. In an embodiment, because of the rotation of the counter, the number displayed on the counter may change as the actuation number increases and may be observed by the patient. After each actuation, the number displayed on the counter changes. Once the predetermined number of actuations is achieved, Protrusion A and Protrusion B will encounter each other, and the counter will be prevented from further rotation. This blocks the atomizer, stopping it from further use. The number of actuations of the device can be counted by the counter.

The nebulizer described above is suitable for nebulizing the aerosol preparations according to the invention to form an aerosol suitable for inhalation.

### EXAMPLES

Materials and Reagents:

50% benzalkonium chloride (referred to as BAC) aqueous solution is commercially available and may be purchased from Spectrum Pharmaceuticals Inc.
Edetate disodium dihydrate is commercially available and may be purchased from purchased from Merck & Co.
Hydrochloric acid is also commercially available and may be purchased from Titan Reagents.

### Example 1

Sample I and Sample II Inhalation Solutions were Prepared as Follows:

50% benzalkonium chloride aqueous solution (50% BAC) and edetate disodium dihydrate according to Table 1 were dissolved in 95 g purified water, and the resulting solutions were adjusted to the target pH with hydrochloric acid as shown in Table 1. Levalbuterol tartrate (R-AS) according to the amounts provided in Table 1 was added to the solutions, and the resulting mixtures were sonicated until completely dissolved. Finally, purified water was added to make 100 g in weight.

TABLE 1
Ingredient Contents of Sample I and Sample II

| | | |
|---|---|---|
| Ingredients | Sample I | Sample II |

| | | | | |
|---|---|---|---|---|
| Levalbuterol tartrate | 20.36 | mg | 20.36 | mg |
| (referred to R-AS) | | | | |
| Edetate Disodium | 11 | mg | 11 | mg |
| Dihydrate | | | | |
| 50% benzalkonium chloride | 20 | mg | 20 | mg |
| aqueous solution | | | | |

| | | |
|---|---|---|
| Hydrochloric acid | To pH 3.0 | To pH 3.4 |
| Purified water | Added to 100 g | Added to 100 g |

### Example 2

Sample III and Sample IV Inhalation Solutions were Prepared as Follows:

50% benzalkonium chloride aqueous solution and edetate disodium dihydrate according to Table 2 were dissolved in 95 g purified water, and the resulting solutions were adjusted to the target pH with hydrochloric acid as shown in Table 2. Levalbuterol tartrate according to the amounts provided in Table 2 was added to the solutions, and the resulting mixtures sonicated until completely dissolved. Finally, purified water was added to make 100 g in weight.

TABLE 2
Ingredient Contents of Sample III and Sample IV

| | | |
|---|---|---|
| Ingredients | Sample III | Sample IV |

| | | | | |
|---|---|---|---|---|
| Lev albuterol tartrate | 10.181 | g | 10.181 | g |
| Edetate Disodium | 11 | mg | 11 | mg |
| Dihydrate | | | | |
| 50% benzalkonium chloride | 20 | mg | 20 | mg |
| aqueous solution | | | | |

| | | |
|---|---|---|
| Hydrochloric acid | To pH 4.0 | To pH 4.5 |
| Purified water | Added to 100 g | Added to 100 g |

### Example 3

Sample V inhalation solution was prepared as follows:

50% benzalkonium chloride aqueous solution and edetate disodium dihydrate according to Table 3 were dissolved in 95 g purified water, and the resulting solution was adjusted to the target pH with hydrochloric acid as shown in Table 3. Levalbuterol tartrate according to the amount provided in Table 3 was added to the solution, and the resulting mixture sonicated until completely dissolved. Finally, purified water was added to make 100 g in weight.

TABLE 3
Ingredient Contents of Sample V

| Ingredients | Sample V | |
|---|---|---|
| Levalbuterol tartrate | 203.6 | mg |
| Edetate Disodium | 11 | mg |
| Dihydrate | | |
| 50% benzalkonium chloride | 20 | mg |
| aqueous solution | | |

| | |
|---|---|
| Hydrochloric acid | To pH 5.0 |
| Purified water | Added to 100 g |

### Example 4

The samples I-V were sprayed using a soft mist inhalation device described in US20190030268. A Malvern Spraytec (STP5311) instrument was used to measure the particle size of the droplets. The results are shown in Table 4.

TABLE 4
Particle Size Distribution of Samples
I-V Using a Soft Mist Inhalation Device

| | |
|---|---|
| Sample Number | Droplet size (μm) |

| | | |
|---|---|---|
| Sample I-V | D10 | 2.1 |
| | D50 | 4.0 |
| | D90 | 8.0 |

### Example 5

Aerodynamic Particle Size Distribution:

An Andersen Cascade Impactor (ACI) was used to determine the particle size distribution of Samples I-V. The test was conducted at a flow rate of 28.3 L/min. Deposition of the active substance on each ACI plate was determined by high performance liquid chromatography. The particle size was expressed in terms of mass median aerodynamic diameter (MMAD) and Geometric Standard Deviation (GSD).

TABLE 5
Aerodynamic Particle Size Distribution

| Parameter | Levalbuterol Tartrate |
|---|---|
| MMAD (μm) | 4.4 |
| GSD | 1.7 |

### Example 6

Administration Using a Soft Mist Inhalation Device

TABLE 6
Ingredient Contents of Sample VI of 200 g Inhalation Solution
Formulation For Administration by Soft Mist Inhalation

| | Sample VI | |
|---|---|---|
| Ingredients | Theoretical dose | |
| R-AS | 470 | mg |
| EDTA | 20 | mg |
| 50% BAC | 40 | mg |

| | |
|---|---|
| target pH | 3.4 |
| water | Add to 200.00 g |

50% benzalkonium chloride aqueous solution and edetate disodium dihydrate according to Table 6 were dissolved in 190 g purified water, and the solution was adjusted to the target pH as shown in Table 6 with hydrochloric acid (HCl). Levalbuterol tartrate (R-AS) according to Table 6 was added to the solution, and then the mixture sonicated until completely dissolved. Finally, purified water was added to a final weight of 200 g.

The aerodynamic particle size distribution was determined using a Next Generation Impactor instrument (NGI). The soft mist inhaler used is disclosed in U.S. 2019/0030268. The soft mist inhaler was held close to the NGI inlet until no aerosol was visible. The flow rate of the NGI was set to 30 L/minute and was operated under ambient temperature and a relative humidity (RH) of 90±2%.

Sample VI was discharged into the NGI. Fractions of the dose were deposited at different stages of the NGI, in accordance with the particle size of the fraction. Each fraction was washed from the stage and analyzed using HPLC. The results are provided in Table 7 below.

TABLE 7
Single Dose Level Distribution and Aerodynamic Particle
Size Distribution of R-AS Inhalation Formulation Sample
VI (2.35 mg/g) Administered by Soft Mist Inhalation

| | | Percentage | Cut-off |
|---|---|---|---|
| R-AS | Dosage | content at | diameter |
| Deposited | (mcg) | all levels | (μm) |
| Throat | 6.63 | 25.93% | |
| Stage 1 | 0.38 | 1.49% | 11.72 |
| Stage 2 | 4.75 | 18.58% | 6.40 |
| Stage 3 | 5.44 | 21.29% | 3.99 |
| Stage 4 | 4.37 | 17.07% | 2.30 |
| Stage 5 | 1.79 | 7.01% | 1.36 |
| Stage 6 | 0.49 | 1.92% | 0.83 |
| Stage 7 | 0.71 | 2.78% | 0.54 |
| Micro-Orifice | 1.01 | 3.94% | |
| Collector (MOC) | | | |
| Theoretical dose (mcg) | 25.97 | | |
| Actual test dose (mcg) | 25.57 | | |
| Recovery rate % | 98.46% | | |
| Fine Particle | 54.01% | | |
| Fraction (FPF) | | | |

Fine Particle Fraction (FPF) is the proportion of fine particle dose in the released dose.

$$F P F = \frac{Mass < 5 μ m}{Mass Total dose} .$$

The larger the FPF value, the higher the atomization efficiency.

### Example 7

Stability Experiment:

Samples VII, VIII, and IX were prepared as follows:
50% benzalkonium chloride aqueous solution and edetate disodium dihydrate according to the amounts provided in Table 8 were dissolved in 190 g purified water, and the resulting solutions adjusted to the target pH values as shown in Table 8 with hydrochloric acid (HCl). R-AS according to the amounts provided in Table 8 was added to the solutions, and the resulting mixtures sonicated until completely dissolved. Finally, purified water was added to a final weight of 200 g.

TABLE 8
Ingredient Contents of Samples VII-IX of
200 g Inhalation Solution Formulation

| | Sample VII | Sample VIII | Sample IX |
|---|---|---|---|
| R-AS | 470 mg | 470 mg | 470 mg |
| EDTA | 20 mg | 20 mg | 20 mg |
| 50% BAC | 40 mg | 40 mg | 40 mg |
| HCl | Adjust to | Adjust to | Adjust to |
| | pH 3.4 | pH 3.1 | pH 3.7 |
| Purified Water | Added to | Added to | Added to |
| | 200.00 g | 200.00 g | 200.00 g |

The obtained solutions were filled into soft mist small bottles and sealed with aluminum foil, and stored at 40° C.±2° C./75%±5% RH.

TABLE 9
Stability of Samples VII-IX

| | R-AS concentration | R-AS |
|---|---|---|
| Sample | (mg/ml) | recovery % |
| Sample VII_0 day | 2.321 | 98.76 |
| Sample VIII_0 day | 2.366 | 100.67 |
| Sample IX_0 day | 2.375 | 101.08 |
| Sample VII_40° C._1 Month | 2.314 | 98.47 |
| Sample VIII_40° C._1 Month | 2.353 | 100.13 |
| Sample IX_40° C._1 Month | 2.345 | 99.79 |
| Sample VII_40° C._3 Months | 2.305 | 98.10 |
| Sample VIII_40° C._3 Months | 2.345 | 99.78 |
| Sample IX_40° C._3 Months | 2.335 | 99.35 |

The impurity analysis method is as follows:

Mobile phase A: 1.30 g sodium heptane sulfonate, dissolve in 1 L water, adjusted to pH to 3.20 with phosphoric acid.
Mobile phase B: Acetonitrile.

### Column: Inertsil ODS-3, 5 μm, 4.6×150 mm, Column Temperature: 35° C.

Flow rate: 1.0 mL/min, Injection Volume: 50 μL, Run Time: 60 minutes, Detection Wavelength: 210 nm
Gradient elution:

| Time (min) | Mobile phase A (%) | Mobile phase B (%) |
|---|---|---|
| 0 | 85 | 15 |
| 10 | 85 | 15 |
| 50 | 65 | 35 |
| 50.1 | 85 | 15 |
| 60 | 85 | 15 |

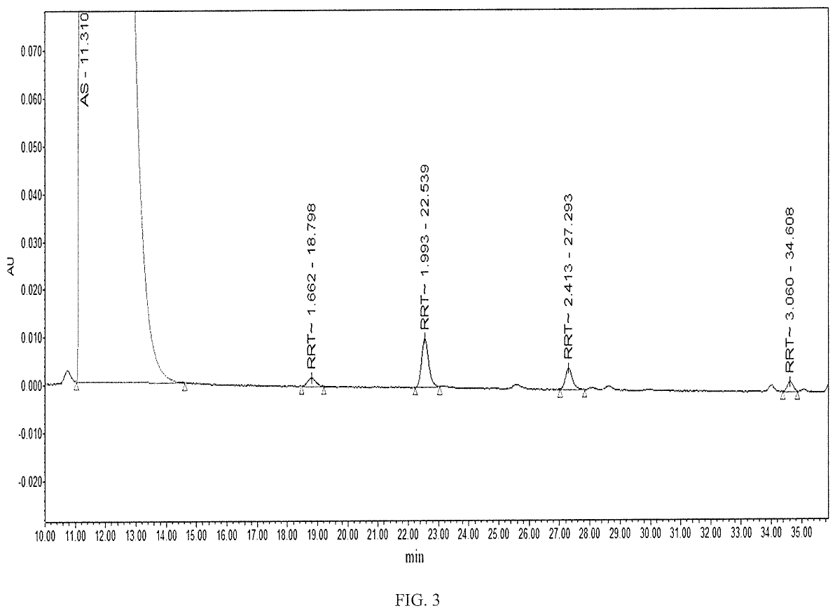

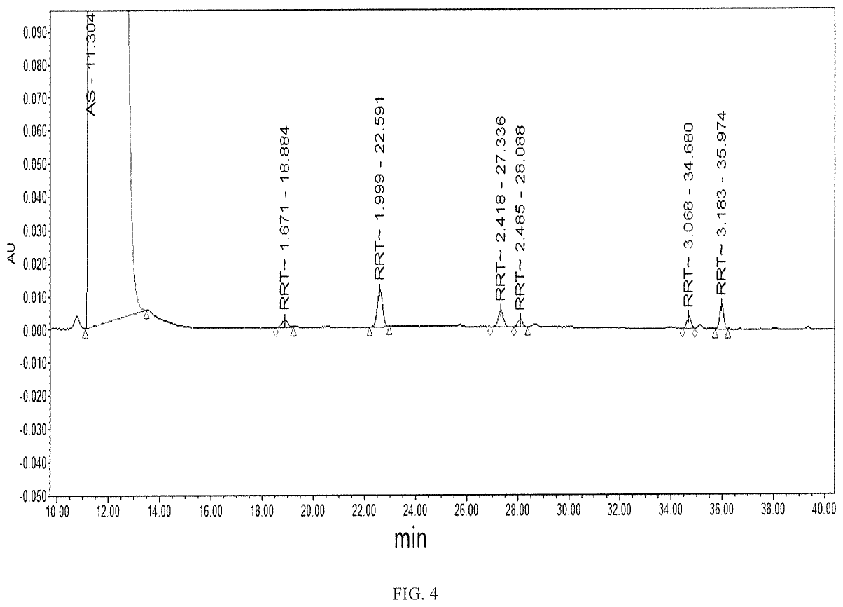

Impurities were analyzed according to the above analysis method. The stability data is shown in Tables 10-12 below. The relative retention time of impurity 1 is 1.66. The relative retention time of impurity 2 is 1.99. The relative retention time of impurity 3 is 2.41. The relative retention time of impurity 4 is 3.05. The relative retention time of impurity 5 is 2.48. The relative retention time of impurity 6 is 3.18. FIGS. 3-4 show HPLC traces demonstrating the relative retention times of the unknown impurities 1-6.

TABLE 10
Stability Results of VII-IX (Conditions: 40° C. ±
2° C./75% ± 5% RH, 0 days)

| | | Sample VII | Sample VIII | Sample IX |
|---|---|---|---|---|
| | RRT | 0 days | 0 days | 0 days |
| Impurity 1 | 1.66 | ND | 0.04 | 0.05 |
| Impurity 2 | 1.99 | 0.21 | 0.21 | 0.21 |
| Impurity 3 | 2.41 | 0.08 | 0.08 | 0.08 |
| Impurity 4 | 3.05 | 0.04 | 0.04 | 0.04 |

| | | | |
|---|---|---|---|
| unknown maximum | 0.21 | 0.21 | 0.21 |
| impurity | | | |
| Total impurities | 0.33 | 0.37 | 0.42 |

TABLE 11
Stability Results of VII-IX (Conditions: 40° C. ±
2° C./75% ± 5% RH, 1 Month)

| | | Sample VII | Sample VIII | Sample IX |
|---|---|---|---|---|
| | RRT | 1 Month | 1 Month | 1 Month |
| Impurity 1 | 1.66 | 0.05 | 0.04 | 0.04 |
| Impurity 2 | 1.99 | 0.21 | 0.21 | 0.21 |
| Impurity 3 | 2.41 | 0.08 | 0.09 | 0.11 |
| Impurity 4 | 3.05 | 0.06 | 0.06 | 0.06 |

| | | | |
|---|---|---|---|
| Unknown maximum | 0.21 | 0.21 | 0.21 |
| impurity | | | |
| Total impurities | 0.50 | 0.55 | 0.56 |

TABLE 12
Stability Results of VII-IX (Conditions: 40° C. ±
2° C./75% ± 5% RH, 3 Months)

| | | Sample VII | Sample VIII | Sample IX |
|---|---|---|---|---|
| | RRT | 3 Months | 3 Months | 3 Months |
| Impurity 1 | 1.67 | 0.05 | 0.05 | 0.05 |
| Impurity 2 | 1.99 | 0.21 | 0.21 | 0.21 |
| Impurity 3 | 2.41 | 0.10 | 0.10 | 0.11 |
| Impurity 5 | 2.48 | 0.04 | 0.04 | 0.04 |
| Impurity 4 | 3.05 | 0.07 | 0.08 | 0.08 |
| Impurity 6 | 3.18 | 0.35 | 0.34 | 0.36 |

| | | | |
|---|---|---|---|
| Unknown maximum | 0.35 | 0.34 | 0.36 |
| impurity | | | |
| Total impurities | 0.82 | 0.82 | 0.85 |

As shown in Tables 8-12, at pH 3.1-3.7 levalbuterol tartrate solutions show good stability. Levalbuterol tartrate solutions ranging from a pH of about 3.1 to about 3.7 are stable for about 3 months at 40° C.±2° C./75%±5% RH.

### Example 8

Comparison Test of Atomization Effect of Different Devices:

The atomization effects of two devices have been compared: 1. The soft mist inhaler disclosed in U.S. 2019/0030268; and 2. An LC-PLUS air compression atomization device.

1. The inhaler disclosed in U.S. 2019/0030268 is made by ourselves.

2. The LC-PLUS air compression atomization device is model Pari TurboBoY, purchased from Pari.

Administration Using a Soft Mist Inhalation Device

The aerodynamic particle size distribution was determined using a Next Generation Impactor instrument (NGI). The soft mist inhaler is disclosed in U.S. 2019/0030268. The soft mist inhaler was held close to the NGI inlet until no aerosol was visible. The flow rate of the NGI was set to 30 L/minute and was operated under ambient temperature and a relative humidity (RH) of 90±2%.

Sample VII shown in Example 7 was discharged into the NGI. Fractions of the dose were deposited at different stages of the NGI, in accordance with the particle size of the fraction. Each fraction was washed from the stage and analyzed using HPLC. The results are provided in Table 13 below.

TABLE 13
Single Dose Level Distribution and Aerodynamic Particle
Size Distribution of R-AS Inhalation Formulation Sample
VII Administered by Soft Mist Inhalation

| | | Percentage | Cut-off |
|---|---|---|---|
| R-AS | Dosage | content at | diameter |
| Deposited | (μg) | all levels | (μm) |
| Throat | 6.63 | 25.93% | |
| Stage 1 | 0.38 | 1.49% | 11.72 |
| Stage 2 | 4.75 | 18.58% | 6.40 |
| Stage 3 | 5.44 | 21.29% | 3.99 |
| Stage 4 | 4.37 | 17.07% | 2.30 |
| Stage 5 | 1.79 | 7.01% | 1.36 |
| Stage 6 | 0.49 | 1.92% | 0.83 |
| Stage 7 | 0.71 | 2.78% | 0.54 |
| Micro-Orifice | 1.01 | 3.94% | |
| Collector (MOC) | | | |
| Theoretical dose (μg) | 25.97 | | |
| Actual test dose (μg) | 25.57 | | |
| Recovery rate | 98.46% | | |
| Fine Particle Fraction | 54.1% | | |
| (FPF) | | | |

Fine Particle Fraction (FPF) is the proportion of fine particle dose in the released dose.

$$F P F = \frac{Mass < 5 μ m}{Mass Total dose} .$$

The larger the FPF value, the higher the atomization efficiency.

Administration Using an LC-PLUS Air Compression Atomization Device

TABLE 14
Ingredient Contents of Sample X of 100 g Inhalation
Solution Formulation for Administration by the
LC-PLUS Air Compression Atomization Device

| Ingredients | Sample X | |
|---|---|---|
| Lev albuterol tartrate | 24.33 | mg |
| NaCl | 900 | mg |
| HCl | Adjust pH to 4.0 | |
| water | 100 | g |

Sample X inhalation solution for administration by the LC-PLUS air compression atomization device was prepared as follows:

NaCl according to the amount in Table 14 was dissolved in 95 g purified water, and the solution was adjusted to the target pH in Table 14 with HCl. Levalbuterol tartrate according to the amount in Table 14 was added to the solution, and the mixture was sonicated until completely dissolved. Finally, purified water was added to a final volume of 100 g.

The aerodynamic particle size distribution was determined using a Next Generation impactor instrument (NGI). The atomization device is the LC-PLUS air compression atomization device. The flow rate of the NGI was set to 15 L/minute and was operated under ambient temperature and a relative humidity (RH) of 90±2%.

Sample X was discharged into the NGI. Fractions of the dose were deposited at different stages of the NGI, in accordance with the particle size of the fraction. Each fraction was washed from the stage and analyzed using HPLC. The results are provided in Table 15 below.

TABLE 15
Single Dose Level Distribution and Aerodynamic
Particle Size Distribution of R-AS Inhalation
Formulation Sample X (0.024 mg/g) Administered
by an LC-PLUS Air Compression Atomization Device

| | | | Percentage | Cut-off |
|---|---|---|---|---|
| R-AS | Dosage | | content at | Diameter |
| Deposited | (μg) | | all levels | (μm) |
| LC Plus device | 400.74 | | 55.38% | |
| Throat | 9.9 | | 1.37% | |
| Stage 1 | 26.63 | | 3.68% | 14.10 |
| Stage 2 | 41.81 | | 5.78% | 8.61 |
| Stage 3 | 63.36 | | 8.76% | 5.39 |
| Stage 4 | 67.57 | | 9.34% | 3.30 |
| Stage 5 | 58.52 | | 8.09% | 2.08 |
| Stage 6 | 36.9 | | 5.10% | 1.36 |
| Stage 7 | 17.21 | | 2.38% | 0.98 |
| MOC | 1.02 | | 0.14% | |
| Theoretical dose | 730 | μg | | |
| Actual test dose | 723.66 | μg | | |

| | |
|---|---|
| Recovery rate | 99.13% |
| FPF | 25% |

Table 15 shows that the fine particle fraction (FPF) is only 25%, which is far lower than the FPF value using the soft mist inhaler with the present invention. When the LC-PLUS air compression atomization device is used to atomize the R-AS solution, a large amount of drug residue remains inside the device and the simulated throat. The medicine remaining inside the device and throat cannot reach the lungs to produce therapeutic effects. The experimental results show that the formulation of the present invention is more effectively atomized by the soft mist device of the present invention than by the LC Plus device.

The R-AS solution formulation of the present invention uses a soft mist device, which has the characteristics of efficient atomization. At the same effective concentration, the R-AS solution formulation of the present invention may be administered at a lower dose than a formulation administered by an LC-PLUS air compression atomization device.

While various embodiments of the present invention have been described above, it should be understood that they have been presented by way of example only and not limitation. For example, the present invention is not limited to the physical arrangements or dimensions. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A liquid, propellant-free pharmaceutical formulation comprising: (a) levalbuterol or a salt thereof; (b) water; (c) a pharmacologically acceptable stabilizer; and (d) a pharmacologically acceptable preservative, wherein the formulation is suitable for administration using a soft mist inhaler.

2. The pharmaceutical formulation of claim 1 comprising levalbuterol tartrate.

3. The pharmaceutical formulation of claim 2, wherein levalbuterol tartrate is present in an amount ranging from about 20 mg/100 g to about 10 g/100 g.

4. The pharmaceutical formulation of claim 2, wherein levalbuterol tartrate is present in an amount ranging from about 200 mg/100 g to about 500 mg/100 g.

5. The pharmaceutical formulation of claim 1, wherein the pharmacologically acceptable preservative is selected from the group consisting of benzalkonium chloride, benzoic acid, sodium benzoate, and a combination thereof.

6. The pharmaceutical formulation of claim 5, wherein the preservative is present in an amount ranging from about 2 mg/100 g to about 1000 mg/100 g.

7. The pharmaceutical formulation of claim 1, wherein the stabilizer is selected from the group consisting of edetic acid, edetate disodium dehydrate, edetate disodium, citric acid, and any combination thereof.

8. The pharmaceutical formulation of claim 6, wherein the stabilizer is present in an amount ranging from about 1 mg/100 g to about 500 mg/100 g.

9. The pharmaceutical formulation of claim 1, comprising a pharmacologically acceptable additive.

10. The pharmaceutical formulation of claim 1, wherein the pharmaceutical formulation has a pH in a range from about 3.0 to about 5.0.

11. A method for administering the pharmaceutical formulation of claim 1 to a patient, comprising forming an inhalable aerosol by using pressure to force a defined amount of the pharmaceutical formulation through a nozzle to nebulize the pharmaceutical formulation.

12. The method of claim 11, wherein the defined amount of the pharmaceutical formulation ranges from about 5 to about 30 microliters.

13. The method formulation of claim 11, wherein the inhalable aerosol has an aerosol D50 of less than about 5 μm.

14. The method of claim 11, wherein the pharmaceutical formulation is administered using an inhaler comprising a blocking function and a counter.

15. A method of treating asthma or COPD in a patient, comprising administering to the patient the pharmaceutical formulation of claim 1.

16. A method of treating asthma or COPD in a patient, comprising administering to the patient the pharmaceutical formulation of claim 2.

17. A method for administering the pharmaceutical formulation of claim 1 to a patient, comprising nebulizing the pharmaceutical formulation in an inhaler, wherein the inhaler includes a blocking function and a counter.

18. The method of claim 11, wherein the patient has asthma or COPD.

19. A device for administering levalbuterol or a salt thereof comprising:
(a) a soft mist inhaler and
(b) a liquid, propellant-free pharmaceutical formulation comprising:
(i) levalbuterol tartrate or a salt thereof present in an amount between about 200 mg/100 g to about 500 mg/100 g;
(ii) water;
(iii) a pharmacologically acceptable stabilizer; and
(iv) a pharmacologically acceptable preservative,
wherein the pharmaceutical formulation has a pH in a range from about 3.0 to about 5.0, and
wherein the formulation is contained in the soft mist inhaler.
